(19) United States
(12) Patent Application Publication
(10) Pub. No.: US 2003/0091638 A1
(43) Pub. Date: May 15, 2003

(54) Polymer matrix having nanopores

(76) Inventors: Emanuel Peled, Even Yehuda (IL); Alexander Vardimon, Tel Aviv (IL); Ronit Bar-Ness Greenstein, Tel Aviv (IL)

Correspondence Address: FISH & RICHARDSON PC, 225 FRANKLIN ST, BOSTON, MA, 02110, US

(21) Appl. No.: 09/950,938

(22) Filed: Sep. 12, 2001

(51) Int. Cl. A61K 9/14

(52) U.S. Cl. 424/486; 424/487

(57) ABSTRACT

A non-toxic polymer matrix comprising pores, a substantial portion of which are essentially less than 50 nm in diameter, the matrix being capable of absorbing and/or transporting an active agent. The matrix comprises:

(a) 5% to 60% by volume of a non-toxic inorganic nanosized powder;

(b) 5% to 50% by volume of a non-toxic polymeric binder; and

(c) optionally, 10% to 90% by volume of an active agent.

The active agent is released from or transported through the matrix at a controlled rate. Also disclosed are an active agent releasing system comprising the non-toxic matrix, and a method for coating a substrate with the matrix.

### FIELD OF THE INVENTION

This invention relates to a polymeric matrix capable of transporting and releasing active substances at a controlled rate.

### BACKGROUND OF THE INVENTION

Millions of people world-wide take drugs and other health-related substances regularly. Often, it is desirable that these substances be released over a predetermined period of time, and not all at once. Such ‘controlled release’, ‘slow-release’ or ‘sustained release’ tablets are well known in the art, and are capable of maintaining effective drug levels for prolonged periods.

The use of fixed orthodontic appliances is a commonly used orthodontic technique for the treatment of malocclusions. In this treatment procedure, small metal attachments, designated as brackets, are bonded onto the teeth. A flexible metal arch-wire is then attached to the brackets, and it is this component that straightens the teeth. Disposable elastomeric rings or rubber bands tie the arch-wire to the bracket.

The orthodontic treatment lasts on the average for 2 years. Each 3-4 weeks the orthodontist reties the same arch-wire, introducing bends in the arch wire or replacing the old arch wire with a new one by changing the type of alloy or the thickness of the arch-wire. In all arch-wire manipulations, the old ER are removed and replaced by new ones, with which the arch-wire is retied.

The Achilles Heel of fixed orthodontic therapy is the high risk of plaque accumulation, resulting in enamel demineralization and development of new carious lesions around the bracket rims. These lesions, which aggravate in the course of fixed orthodontic treatment, sometimes begin in the form of white spots. In addition to the possible development of caries and subsequent tooth decay, this constitutes an aesthetic problem, since the clinical management of white spot lesions is still unresolved. A longitudinal study has shown a significant modification of the oral microbiota in patients with fixed appliances, suggesting high risk for gingivitis and periodontitis during orthodontic therapy. These side effects could have an irreversible consequence to the dental health of the patient.

Many clinical trials and other research studies have shown the efficacy of antibacterial drugs such as chlorhexidine as a plaque inhibitory agent, by suppressing oral mutans Streptococci levels and gingivitis for long periods. In some studies, chlorhexidine mouthwashes have been used in orthodontic patients for plaque control and oral hygiene maintenance, whereas others used chlorhexidine varnish for the same purpose. Since efficacy of mouth rinsing relies on patient compliance, this manner of application is unreliable.

Very often, caries initiation occurs at contact point areas between teeth (interproximal areas). In its incipient state, the progress of a carious process can be inhibited if the cariogenic bacteria are eliminated and demineralized enamel areas undergo remineralization.

WO 99/44245 discloses an ion conducting matrix comprising an inorganic powder having a good aqueous electrolyte absorption capacity, a polymeric binder that is chemically compatible with an aqueous electrolyte, and an aqueous electrolyte. The inorganic powder comprises essentially sub-micron particles. The matrix may be used in the manufacture of electrochemical cells.

U.S. patent application Ser. No. 09/484,267 filed Jan. 18, 2000 discloses a fuel cell having a solid electrolyte membrane with an anode side and a cathode side. The membrane is a proton conducting membrane having pores smaller than 30 nm, and comprising an electrically nonconductive inorganic powder having a good acid absorption capacity, a polymeric binder that is chemically compatible with acid, oxygen and fuel and an acid or aqueous acid solution. The inorganic powder is comprised of essentially nano-sized particles.

U.S. Pat. No. 5,674,067 discloses a flavored orthodontic elastic band comprising an inner elastic band member and an outer porous coating layer carrying a flavoring substance that is released upon exposure to saliva, for a predetermined time corresponding to the effective life of the inner elastic band. The porous elastic layer comprises an orally acceptable elastomeric material such as rubber, thermoplastic elastomers and blends thereof. An effective amount of a swelling polymer can also be included in the outer elastic layer.

### SUMMARY OF THE INVENTION

It is an object of the present invention to provide a matrix having nano-sized pores and capable of releasing molecules contained therein in a controlled fashion.

It is another object of the invention to provide substrates coated with this matrix in the form of a membrane.

It is a still further object of the invention to provide a matrix coating a substrate which comprises an active substance.

In one aspect of the invention, there is provided a non-toxic polymer matrix comprising pores, a substantial portion of which are essentially less than 50 nm in diameter, the matrix being capable of absorbing and/or transporting an active agent. The matrix comprises:

(a) 5% to 60% by volume of a non-toxic inorganic nanosized powder;

(b) 5% to 50% by volume of a non-toxic polymeric binder; and

(c) optionally, 10% to 90% by volume of an active agent;

wherein the active agent is released from or transported through the matrix at a controlled rate.

In the present specification, the term matrix includes within its scope the substance that comprises the whole of a substrate, such as a pharmaceutical tablet, as well as the substance which coats a substrate, such as the coating enveloping a pill or a medical device. In the later case, the term membrane may also be used.

The polymeric matrix according to the invention may be used either as a sponge which absorbs various substances and then releases them over time, or as a coating for a substrate. When used as a coating (membrane), the matrix may be used in two manners:

1. for controlling release and transport of substances previously absorbed by the coated substrate; or

2. the matrix itself may contain therein various substances to be released in a controlled manner.

All of the components of the matrix must be non-toxic. In the present specification, the term “non-toxic” refers to substances which do not have a pathological effect when coming into contact with an organism for which they are intended, as part of the matrix and at concentration levels required for forming the matrix. Preferably, the components are also biocompatible.

Furthermore, the matrix and, in particular, the inorganic powder must be capable of absorbing and/or transporting the active agent. Preferably, the matrix is capable of absorbing the active agent at a ratio of >1:20 active agent: matrix (w/w).

The inorganic powder consists of nanosized grains, that is, of a size essentially less than 100 nm. Examples of an inorganic powder which may be used in the matrix of the invention include, but are not limited to, SiO₂, MgO, Al₂O₃, hydroxides and oxy-hydroxides of Al and Mg, and any combinations thereof.

Examples of an polymeric binder which may be used in the matrix of the invention include, but are not limited to, polyvinylidene fluoride (PVDF), hexafluoropropylene, poly(methyl methacrylate), poly(vinylchloride), poly(vinylfluoride), Kel F™, polyvinyl alcohol (PVA), latex, rubber and any combinations thereof.

The active agent may be any pharmaceutical or orally ingested substance such as an antibacterial substance, a drug for either internal or external use, a hormone, a vitamin or a food product. In the present specification, the term food product also includes any substance used in the preparation of a food product, such as food additives. The active agent may also be a cosmetic substance such as a perfume. The active agent may be applied not only orally but also through a surface of the body, either externally (i.e. topically, e.g. on the skin or the tooth surface) or internally (e.g. subcutanously, intravenously, intraperitoneally, intramuscularly or intrathecally). The active agent is either stored in the nano-sized pores or transported through the pores from the coated substrate in which it is contained.

The pore size may be controlled by the type and size of the inorganic powder and by the relative proportions of the matrix components, and may be conformed to the size of the active agent. In general, increasing the amount of the inorganic powder leads to an increase in the average pore size. Preferably, the pores are essentially less than 4 nm in diameter. Most preferably, the pores are essentially less than 1.5 nm in diameter.

In a second aspect of the invention there is provided an active agent releasing system comprising a non-toxic matrix according to the invention deposited on a substrate.

Non-limiting examples of substrates which may be coated with a membrane according to the invention include a pharmaceutical tablet, a food product or a medical device. Specific examples include:

controlled release of flavors, minerals or vitamins in food products (iron and vitamins in breakfast cereal);

controlled release of pharmaceutical agents from medical devices (bandages, dental implants);

controlled release of antibacterial substances from an elastic orthodontic device such as an elastomeric ring, a ring separator (also known as an elastic orthodontic separator), plastic chain, plastic ligature, ligature ringlets, elastomeric ligature, separator, elastic ligature, modules, ligature modules, power chain and elastics;

controlled release of remineralization agents (e.g. fluorides) from a ring separator (also known as an elastic orthodontic separator) which may be placed between teeth at areas of caries initiation;

controlled release of hormones and drugs from pills.

In a third aspect of the invention, there is provided a method for coating a substrate with a matrix according to the invention. The method comprises:

(a) suspending 5% to 60% by volume of an inorganic non-toxic nanosized powder, 5% to 50% by volume of a non-toxic polymeric binder and optionally 10% to 90% by volume of an active agent in an organic solvent;

(b) applying the suspension to the substrate so as to coat it;

(c) evaporating the solvent; and, if the active agent was not included in step (i),

(d) incubating the coated substrate with an active agent.

The membrane may be formed by preparing a suspension of the inorganic powder and polymeric binder, preferably in an organic solvent which may include more than one type of solvent. The substrate may then be dipped in the suspension and subsequently dried. Alternatively, the suspension may be sprayed onto the substrate. The active agent may be applied to the membrane either together with the suspension or subsequent to the coating. Preferably, the substrate is soaked in the active agent.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 is a graph showing the rate of ascorbic acid release from Vitamin C tablets prepared according to one embodiment of the invention;

FIG. 2 is a graph showing the rate of acetylsalicylic acid release from aspirin tablets prepared according to one embodiment of the invention.

### DETAILED DESCRIPTION OF THE INVENTION

### I. Coated Elastomeric Rings (ER)

### Experiment 1

Preparation and Testing of the ER:

Different types of disposable ER used in orthodontic treatment (with latex: Ortho Organizers; without latex: Quik Stik Unitek Alastik) and of different sizes were used. The ER were coated by a nanometric polymeric matrix according to one embodiment of the invention, in order to increase their liquid absorbance capacity. In brief, the polymeric material composing the ER=latex/non latex polyurethane (acting as the polymeric binder) and nanosize ceramic amorphous powder of fumed silicon dioxide, 15 nm particle size 99.8% (130, Degussa) were suspended in cyclopentanone. ER was then dipped in the suspension. Following coating, they were soaked for 24 hours in a 1% solution of the antibacterial substance Chlorhexidine.

The slow releasing capability of the coated ER was measured in an in vitro antibacterial assay, as follows:

One. Bacterial lawns of Streptococcus mutans on brain-heart infusion agar plates were prepared.

Two. The pre-soaked ER (after drying) were applied to the bacterial lawns.

Three. The plates were incubated for 48 hours in anaerobic conditions at 37° C.

Four. The zone of bacterial growth inhibition was measured and compared between the different ER and antibacterial agent solutions.

Five. The ER were removed and re-applied onto new bacterial lawns on the same side of the ER.

Six. Incubation and measurement were repeated as described above (steps c, d & e).

Seven. This procedure (steps e and f) was repeated until an antibacterial effect was no longer detected.

Results:

The ability of the different types of ER to inhibit bacterial outgrowth following soaking in the antibacterial solutions was tested as described above. The resulting data are presented in Table 1:

TABLE 1
Antibacterial effect of coated ER

| | | | Maximal | |
|---|---|---|---|---|
| No. | ER size | Coating | Application No. | Days |
| 1 | large | with | 12 | 34 |
| 2 | small | with | 6 | 28 |
| 3 | small | with | 6 | 18 |
| 4 | large | none | 2 | 6 |
| 5 | small | none | 1 | 4 |
| 6 | small | none | 1 | 2 |

Maximal Application No. - the number of the last application of the elastic band which still gave bacterial inhibition;
Days - the number of days passed since the beginning of the experiment (immediately after taking the ER out of soaking in the antibacterial agent);

Conclusions:

The polymeric matrix coating enhanced the efficiency of the bacterial inhibition capability of the ER. Coating No. 1 on large ER was the most efficient, probably due to their larger absorbance capacity as compared to small ER 2 and 3.

### Experiment 2

Elastic Strength Test of Coated ER

The elastic properties of the coated ER were tested, in order to compare them with regular uncoated ER. Shearing force was applied to ER (coated or regular of the same kind), by a testing machine (Instron Model 4502, High Wycombe, Buckinghamshire, UK) at a cross head speed of 0.5 mm/min up to failure.

The coated ER were checked under a video-microscope before and after stretching in the testing machine, in order to examine the coating under the orthodontic stretching pressure. The stretching strength was identical to that which occurs in the mouth.

Results:

The coated ER showed a similar elastic strength pattern to that of the regular ones. The coating remained undamaged after stretching in the Instron apparatus.

Conclusions:

The elastic strength pattern of the coated ER suggests that the coating is suitable for orthodontic use. The stretching did not damage the coating. Thus the coated ER are expected to be efficient under clinical orthodontic conditions as a slow releasing device of antibacterial or other active agents.

### II. Controlled Drug Release

### Experiment 1

Three types of tablets were used in order to test the rate of drug release: vitamin C (ascorbic acid) (500 mg—Rekah, Ltd., Israel), Aspirin (acetyl salicylic acid) (100 mg—Goodmade, Inc.) and an antibiotic (Rafapen—Rafah Laboratories, Ltd., Israel). The tablets were coated with a porous membrane according to one embodiment of the invention in order to obtain slow release of the active agent.

Membrane Preparation:

A mixture of measured amounts of the polymeric binder Kynar poly(vinylidene fluoride) (PVDF) 2801-00 (ELF Autochem) and nanosize ceramic amorphous powder of fumed silicon dioxide, 15 mm particle size 99.8% (130, Degussa) were suspended in cyclopentanone. Both the binder and the powder are non-toxic in vivo. Three different PVDF:SiO₂ ratios were checked as detailed in Table 2. The ratio binder:powder determines properties of the membrane, such as flexibility, porosity, and mechanical stability. In general, flexibility and mechanical strength increase and porosity decreased with the increase in the polymer content of the membrane.

TABLE 2

| | PVDF:SiO₂ | PVDF:SiO₂ |
|---|---|---|
| Tablet No. | (W/W) | (V/V) |
| 1 | 10:2.2 | 34:6 |
| 2 | 10:2.5 | 32:8 |
| 3 | 10:4 | 30:10 |
| 4 | 10:6 | 24:12 |

The tablets were coated by dipping them in the membrane suspension and were air-dried for a few minutes on a stand of three sharp pins or on a glass plate. Each tablet was coated two to three times. The final thickness of the coating was found to be in the range of 5-15 μm.

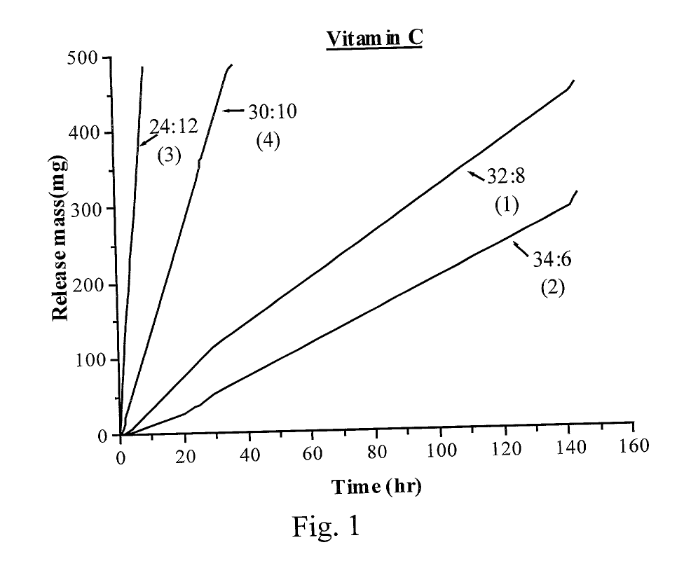

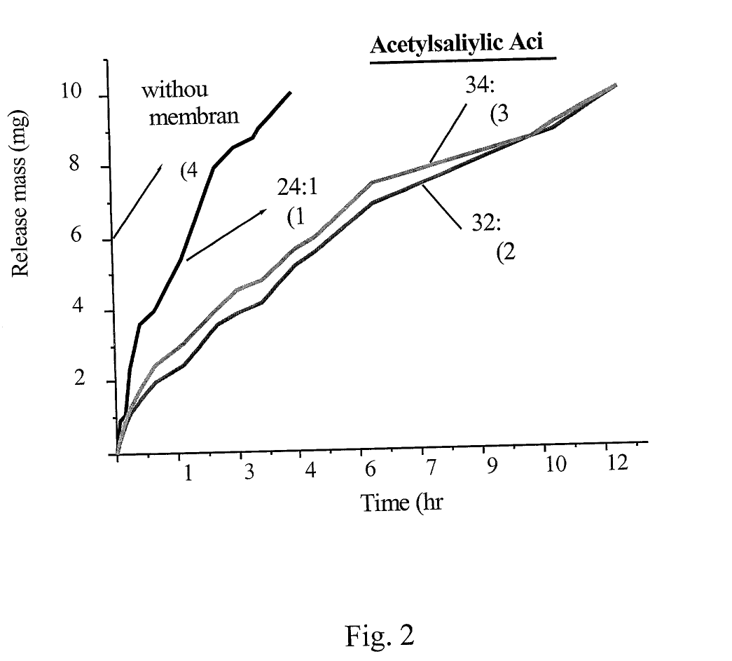

The rate of drug release from the tablets into pure water at 23±3° c. was measured. Vitamin C and Aspirin were determined from time to time by titration with 0.1M sodium hydroxide (phenolphthalein indicator). The results are presented in FIGS. 1 and 2.

It was found that the rate of drug release depends on the PVDF:SiO₂ ratio. As can be seen from FIGS. 1 and 2, the rate of release of active agent is directly proportional to the content of SiO₂ content in the membrane. It has been previously found that both the typical pore size and membrane porosity decrease with a decrease in SiO₂ content. The complete release of the drugs took place between 20 and 200 hours, which is a practical range for controlled release of drugs. In addition, the rate of release does not change much with time or as a function of the residual amount of the drug in the tablet.

### Experiment 2

The purpose of this experiment was to test the drug release rate into distilled water (at room temperature), of several types of membrane-coated Rafapen tablets prepared as described above in Experiment 1, as compared to a non-coated Rafapen tablet (control).

In Vitro Antibacterial Assay:

One. Bacterial lawns of Streptococcus mutans on brain-heart infusion agar plates were prepared.

Two. Each Rafapen tablet was put into a separate test tube containing distilled water (3 ml). The test tubes were inserted into a test tube shaker at room temperature. Samples (0.01 ml) were taken at several time points (0, 1 h, 2 h, 3 h, 24 h, 48 h and 72 h) from the aqueous phase and applied to the bacterial lawns.

Three. The plates were incubated for 48 h under anaerobic conditions at 37° C.

Four. The zone of bacterial inhibition was measured and compared between the different membrane-coated and non-coated Rafapen tablets.

Results

The drug release rate was determined by measuring the antibacterial activity (zone of inhibition of the bacterial outgrowth) of the Rafapen tablets at the different time points. The data are presented in Table 3.

TABLE 3
Antibacterial effect versus time.
Results are expressed as inhibition diameters (cm)

| PVDF to SiO₂ | | | | | | | |
|---|---|---|---|---|---|---|---|
| (V:V) [coating | | | | | | | |
| thickness (μm)] | Time 0 | 1 h | 2 h | 3 h | 24 h | 48 h | 72 h |
| Non-coated | 4.0 | 5.25 | 5.15 | 5.25 | 4.75 | 5.5 | 5.5 |
| 34:6 [7] | 0 | 0 | 0 | 1.7 | 3.5 | 4.2 | 5.75 |
| 34:6 [8] | 0 | 0 | 0 | 1.3 | 3.3 | 4.8 | 5.75 |
| 32:8 [10] | 0 | 1.2 | 2.4 | 2.6 | 5.0 | 5.5 | 5.5 |

Conclusions

The coating decreases the release rate of the antibiotic in the coated tablets as compared to the control tablet. Membrane coating ratio 34:6 showed a slower release rate as compared to the 32:8 ratio and to the non-coated control Thus, the membranes with the higher SiO₂ content (32:8 [10 μm]) had higher porosity as well as a higher drug release rate. Membrane coating 34:6 [8 μm] showed a slightly slower release rate, possibly due to a thicker coating as compared to 34:6 [7 μm]. The experiment was terminated after 72 h because all coated tablets reached the maximal inhibition zone as compared to the non-coated tablet.

The results of this experiment show that coatings prepared according to the invention have a potential use as controlled release mediators, not only in orthodontics but also in other areas such as medical, cosmetics, veterinary, agriculture, etc.

1. A non-toxic polymer matrix comprising pores, a substantial portion of which are essentially less than 50 nm in diameter, said matrix being capable of absorbing and/or transporting an active agent, said matrix comprising:
(a) 5% to 60% by volume of a non-toxic inorganic nanosized powder;
(b) 5% to 50% by volume of a non-toxic polymeric binder; and
(c) optionally, 10% to 90% by volume of an active agent;
wherein said active agent is released from or transported through said matrix at a controlled rate.

2. A matrix according to claim 1 in the form of a membrane deposited on a substrate.

3. A matrix according to claim 2 wherein said membrane comprises said active agent.

4. A matrix according to claim 2 wherein said substrate comprises said active agent.

5. A matrix according to claim 1 wherein said matrix is capable of absorbing said active agent at a ratio of >1:20 active agent:matrix (w/w).

6. A matrix according to claim 1 wherein said non-toxic inorganic powder consists of grains of a size essentially less than 100 nm.

7. A matrix according to claim 1 wherein said non-toxic powder is selected from the group consisting of SiO₂, MgO, Al₂O₃, hydroxides and oxy-hydroxides of Al and Mg, and any combinations thereof.

8. A matrix according to claim 1 wherein said polymeric binder is selected from the group consisting of polyvinilydene fluoride (PVDF), hexafluoropropylene, poly(methyl methacrylate), poly(vinylchloride), poly(vinylfluoride), Kel F™, polyvinyl alcohol (PVA), latex, rubber and any combinations thereof.

9. A matrix according to claim 1 wherein said active agent is located in said pores.

10. A matrix according to claim 1 wherein said active agent is an antibacterial substance, a drug, a remineralization agent, a vitamin, a hormone, a cosmetic substance or a food additive.

11. A matrix according to claim 1 wherein said pores are essentially less than 4 nm in diameter.

12. A matrix according to claim 11 wherein said pores are essentially less than 1.5 nm in diameter.

13. An active agent releasing system comprising a non-toxic matrix according to claim 1 deposited on a substrate.

14. A system according to claim 13 wherein said substrate is a pharmaceutical tablet, a food product or a medical device.

15. A system according to claim 14 wherein said medical device is an elastic orthodontic device.

16. A controlled-release tablet coated with a non-toxic membrane according to claim 2.

17. A food product coated with a non-toxic membrane according to claim 2.

18. A cosmetic product coated with a non-toxic membrane according to claim 2.

19. A medical device coated with a non-toxic membrane according to claim 2.

20. A medical device according to claim 19 which is an elastic orthodontic device.

21. A medical device according to claim 20 selected from the group consisting of elastomeric ring, ring separator, elastic orthodontic separator, plastic chain, plastic ligature, ligature ringlets, elastomeric ligature, separator, elastic ligature, modules, ligature modules, power chain and elastics.

22. A method for coating a substrate with a matrix according to claim 1 comprising:
(a) suspending 5% to 60% by volume of an inorganic non-toxic nanosized powder, 5% to 50% by volume of a non-toxic polymeric binder and optionally 10% to 90% by volume of an active agent in an organic solvent;
(b) applying said suspension to said substrate so as to coat it;
(c) evaporating the solvent;
and, if the active agent was not included in step (i),
(d) incubating said coated substrate with an active agent.

23. A method according to claim 22 wherein the ratio of said binder to said powder ranges from 1:12 to 10:1 (V/V).

24. A method according to claim 22 wherein said active agent is added in a pure state, in a mixture or in a solution.
